(19) United States
(12) Patent Application Publication
(10) Pub. No.: US 2025/0165922 A1
(43) Pub. Date: May 22, 2025

(54) SYSTEM, APPARATUS AND METHODS FOR COUNTING AND DETECTING SURGICAL SPONGES

(71) Applicant: Augustine Biomedical and Design, LLC, Eden Prairie, MN (US)

(72) Inventor: Scott D. Augustine, Deephaven, MN (US)

(21) Appl. No.: 19/030,395

(22) Filed: Jan. 17, 2025

Related U.S. Application Data
(63) Continuation of application No. 18/505,821, filed on Nov. 9, 2023.
Application No. 18/505,821 is a continuation of application No. 14/615,678, filed on Feb. 6, 2015, now abandoned.
(60) Provisional application No. 61/936,508, filed on Feb. 6, 2014.

(51) Int. Cl. G06Q 10/0875; A61B 50/37; A61B 90/00; A61B 90/98; G06K 19/08

(52) U.S. Cl. CPC G06Q 10/0875; A61B 50/37; A61B 90/98; G06K 19/08; A61B 2050/375; A61B 2090/0804; A61B 2090/0805

(57) ABSTRACT

System, methods and apparatus related to surgical sponges and surgical sponge counting and detection systems. The system, methods and apparatus may include features such as a Radio Frequency Identification (RFID) scanner adapted to detect entry and exit of a surgical sponge RFID identifier associated with each of a plurality of surgical sponges. In addition, an Electronic Article Surveillance (EAS) scanner may be included that is adapted to detect the presence of a surgical sponge having an EAS identifier associated with each of the plurality of surgical sponges. A control circuit may be configured to determine and provide an indication that all the sponges have been removed from the patient, or that one or more sponges still remain in the patient.

### CROSS-REFERENCE TO RELATED APPLICATIONS

This application is a continuation of U.S. patent application Ser. No. 18/505,821, filed on Nov. 9, 2023, which is a continuation of U.S. patent application Ser. No. 14/615,678, filed Feb. 6, 2015, which claims priority to U.S. Patent Application 61/936,508, filed Feb. 6, 2014, each of which is incorporated herein by reference in its entirety.

### TECHNICAL FIELD

This disclosure relates to counting and detection of surgical sponges.

### BACKGROUND

Radio Frequency Identification (RFID) of many types of objects is well known in the arts, including the counting of surgical sponges outside the human body and the detection of retained surgical sponges inside the human body. In general, RFID consists of a radio-frequency emitter antenna that energizes a non-powered identifier in an RFID tag. The identifier utilizes the RF energy transmitted through the air to power an RF response signal, transmitting data that can be detected by a receiver antenna in the reader device.

In general, RFID is available in three categories: Electronic Article Surveillance (EAS), High Frequency RFID (HF-RFID), and Ultra-High Frequency RFID (UHF-RFID). Features and characteristics of the three categories of RFID will now be described.

The first category of RFID, Electronic Article Surveillance Systems (EAS) are available in several different types including: magnetic (also known as magneto-harmonic), acousto-magnetic (also known as magnetostrictive), radio frequency (also known as low frequency) and microwave. EAS utilizes a relatively low frequency range of RF signal (10 Hz-10 MHz), to energize a ferromagnetic strip or an antenna coupled to a capacitor. These simple and inexpensive RFID devices emit a simple signal that does not include any identifying information. EAS technology is useful for detection (presence or absence) only. It is commonly used to prevent shoplifting or pilferage of books from libraries. The low frequency signal is capable of traversing through the tissue of the human body. Therefore, the EAS technology can be used only for detection which can include detection of surgical sponges retained within the human body.

The second category of RFID, High Frequency RFID (HF-RFID) may utilize a mid-range frequency of 13.6 MHz. These identifiers are generally made by connecting an Integrated Circuit (IC) chip to an antenna. The antenna may or may not include windings around a ferrite core. The IC of the HF-RFID identifier is capable of being programmed to both remember a variety of alpha-numeric data and to broadcast that data so that it can be interpreted by the reader device. The power to the IC chip is supplied by the RF energy captured by the antenna. HF-RFID can be used for specific identification and counting of objects including surgical sponges. HF has a relatively short broadcast range through the air but is also capable of traversing through the tissue of the human body. HF-RFID can therefore be used for both counting and detection of surgical sponges retained within the human body.

The third category of RFID, Ultra-High Frequency RFID (UHF-RFID) may utilize a relatively high frequency of 902-928 MHz. Similar to HF, these identifiers are made by connecting an Integrated Circuit (IC) chip to an antenna. Because of the higher frequency, the UHF IC chips are smaller and cheaper than the HF chips. Similar to HF, the IC of the UHF-RFID identifier is capable of being programmed to both remember a variety of alpha-numeric data and to broadcast that data so that it can be interpreted by the reader device. The power to the IC chip is supplied by the RF energy captured by the antenna. UHF-RFID can be used for specific identification and counting of objects including surgical sponges. UHF has a relatively long broadcast range through the air but is not capable of traversing through the tissue of the human body. UHF-RFID can therefore be used for counting but not for detection of surgical sponges retained within the human body.

Radio Frequency Identification (RFID) and Electronic Article Surveillance (EAS) antennas incorporated into mattress overlays have been used for surgical sponge detection inside the human body. Examples of these devices are disclosed in U.S. Patents: Fleck U.S. Pat. No. 8,479,989; Fleck U.S. Pat. No. 8,256,674; Fleck U.S. Pat. No. 8,576,076; Blair U.S. Pat. No. 6,026,818 and Fabian U.S. Pat. No. 5,057,095. These systems have been shown to be unreliable, failing to detect retained surgical sponges 1-2% of the time. Therefore, detection of retained surgical sponges by itself cannot be relied upon to be accurate. In addition, the mattress overlays housing these antennas are inconvenient and require extra cleaning.

High Frequency-Radio Frequency Identification (HF-RFID) has been used to identify and count surgical sponges outside the human body for many years. Examples of these devices are disclosed in U.S. Patents: Morris U.S. Pat. No. 8,576,076; Morris U.S. Pat. No. 8,279,068; Morris U.S. Pat. No. 8,105,296; Morris U.S. Pat. No. 6,998,541; Morris U.S. Pat. No. 5,650,596; Morris U.S. Pat. No. 5,923,001 and Everett U.S. Pat. No. 5,491,468. HF-RFID sponge counting has been shown the have a failure rate as high as 1% and is prohibitively expensive.

Considering the severity of the infections that result from retained surgical sponges, a 1-2% failure rate is simply unacceptable. Therefore, hospitals using these systems still have the nurses manually counting the sponges as a backup to the HF-RFID count or the EAS detection systems. Since retained surgical sponges are defined as a “never event”—events that should never occur, automated counting systems will only replace human counting when they are nearly 100% reliable.

All of the RFID technologies described (EAS, HF-RFID and UHF-RFID) have a measurable failure rate, meaning that they fail to detect a retained surgical sponge that has been inadvertently left inside the patient before closing the incision. Each of these technologies is arguably approaching the limits of its reliability. There is a need for a more reliable surgical sponge counting system to replace or reliably augment the traditional sponge counting currently done by the nurse.

### SUMMARY

“Backup systems” for preventing errors are well known in the art of surgical medicine. However, in virtually all instances, the “backup system” is engaged at the time the primary system fails. Rather than the traditional “backup” methodology, the illustrative embodiments described herein uniquely use two dissimilar RFID systems simultaneously operating in parallel in order to mathematically reduce the rate of failure. The result is a geometric 100× or more improvement in the reliability of surgical sponge detection and counting.

In some embodiments, two RF detection technologies are advantageously used in parallel to detect retained sponges. For example, a tag on a sponge may include a sophisticated and relatively expensive UHF-RFID identifier for detection and counting outside the body and a very inexpensive EAS identifier for detection within the body. These two RF detection technologies are essentially operating in parallel. Since each technology has different failure modes or fails for different reasons, the failure rate of the combined system is approximately the failure rate of each technology multiplied together. For example, if each failed 1% of the time, the combined failure rate would be 0.01%. This uniquely leads to failure rates that are orders of magnitude less than can be achieved with any single sensing technology.

In some embodiments a single HF-RFID identifier could be interrogated by two separate readers creating a parallel detection system. For example, a sophisticated reader/counter may be used to detect and count sponges outside the body and a much less sophisticated detector without counting and identification capabilities may be embedded in the underbody support. Both of these detectors would be interrogating the same HF-RFID identifier but operating in parallel.

This reliability rate can be even further improved by adding an algorithm to analyze the input data from both RFID sources. The two RFID technologies operating in parallel uniquely offer a degree of reliability that could never be achieved with any single system.

In some embodiments, the design of the tags and sponges that simultaneously incorporate UHF-RFID and EAS identifier, must minimize the chances of the adjacent tags from “coupling” and canceling each other out, thus becoming undetectable. The two RF identifiers may be maximally separated geographically, either on the same tag or on the same sponge. The two RF identifiers may also be maximally separated in operating frequency. For example, the frequency difference between EAS and UHF is greater than between EAS and HF. Therefore the combination of EAS and UHF may be used in some embodiments to prevent interference between the adjacent identifiers.

In some embodiments, the antenna for the EAS detector for detection within the body or the unsophisticated HF detector for detection within the body, is embedded in the underbody support. Embedding the antenna in the underbody support (e.g., mattress) may be more convenient because it can eliminate the mattress overlay that requires extra cleaning and also can eliminate the user variability caused by hand-held antennas and the practice of “wanding.” In some embodiments, it also allows RF absorbing polypyrrole heater material to absorb stray RF signals, improving the detection and clarity of the signal emitted from the tag.

In some embodiments, polypyrrole material and/or metal foils may also be used to surround the antennas in the freestanding reader/counter and discarded sponge bucket. The purpose of the polypyrrole material and/or metal foils is to reduce stray RF detection, to decrease the broadcast of RF signals throughout the room, and to remove the discarded counted sponges from the ongoing interrogation process.

In some embodiments, the radiolucency of the underbody support is maintained by either restricting the antennas to the periphery of the support or using radiolucent conductors.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a block diagram illustrating an embodiment of a counting system.

FIG. 2 is a top view of first embodiment of a tag including RFID and EAS identifiers.

FIG. 3 is a top view of second embodiment of a tag including RFID and EAS identifiers.

FIG. 4 is a top view of third embodiment of a tag including RFID and EAS identifiers.

FIG. 5 is a top view of fourth embodiment of a tag including RFID and EAS identifiers.

FIG. 6 is a top view of an embodiment of a surgical sponge including RFID and EAS identifiers.

FIG. 7 is a top view of another embodiment of a surgical sponge including RFID and EAS identifiers.

FIG. 8 is a top view of fifth embodiment of a tag including RFID and EAS identifiers.

FIG. 9 is a top view of sixth embodiment of a tag including RFID and EAS identifiers.

FIG. 10 is a flow chart illustrating and embodiment of a decision tree for detecting and rectifying a sponge miscount.

FIG. 11 is a perspective view of a surgical field including an embodiment of an underbody support for detecting the presence of a sponge within a patient.

FIG. 12 is a perspective view of a surgical field including another embodiment of an underbody support for detecting the presence of a sponge within a patient.

FIG. 13 is a perspective view of a surgical field including another embodiment of an underbody support for detecting the presence of a sponge within a patient.

FIG. 14 is a perspective view of an embodiment of a counting device of the counting and detection system of FIG. 1a.

### DETAILED DESCRIPTION

The following detailed description is exemplary in nature and is not intended to limit the scope, applicability or configuration of the invention in any way. Rather, the following description provides practical illustrations for implementing illustrative embodiments of the invention. Examples of constructions, materials, dimensions, and manufacturing processes are provided for selected elements; all other elements employ that which is known to those of ordinary skill in the field of the invention. Those skilled in the present art will recognize that many of the noted examples have a variety of suitable alternatives.

The invention provides a surgical sponge having an absorbent body and one or more identifiers attached to or embedded within the sponge. The invention also provides a surgical sponge counting and detection system for counting surgical sponges, and a method for counting and detection of surgical sponges. The present invention may reduce the likelihood that a sponge will remain in a patient after surgery and may reduce the time involved in accounting for sponges during surgery.

In some embodiments, when sponges enter a detection field, identifiers in the sponges 2 may emit responsive data thereby enabling the system, in the manner to be described hereinafter, to identify the specific sponge which is in the field. The identifier may be any suitable identifier such as one with an integrated circuit and an antenna (e.g. a copper wire or printed circuit antenna). This may enable the system to identify the specific type of sponge, to count the objects and to confirm the presence of the object on an individual basis or for a plurality of objects.

The identifier may include a numerical identifier, an alphabetical identifier, an alpha-numeric identifier, or other type of identifier. The identifiers on a sponge may also be programmed with information including but not limited to: a specific serial number, type, size and weight of the sponge, the number of sponges in a pack, the sponge manufacturer and manufacturing dates. The identifiers on a sponge may also be programmed with information about the sponges treatment history, such as, for example, sterilization of the sponge, which is information that may be added at the time of sterilization. The identifiers on the sponges may also be programmed as they are read.

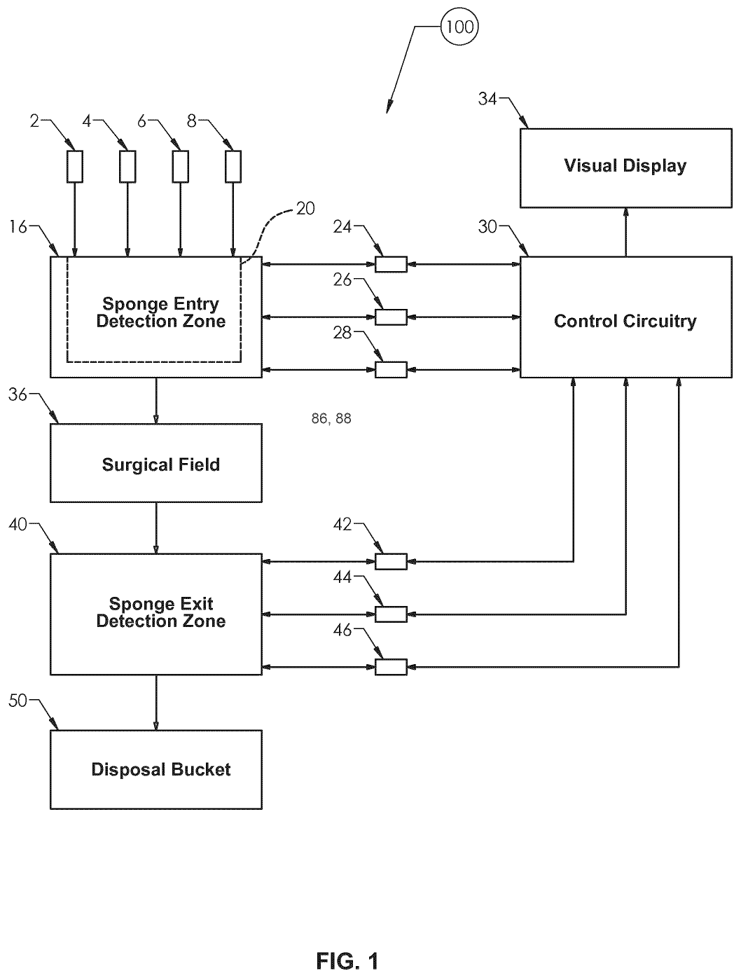

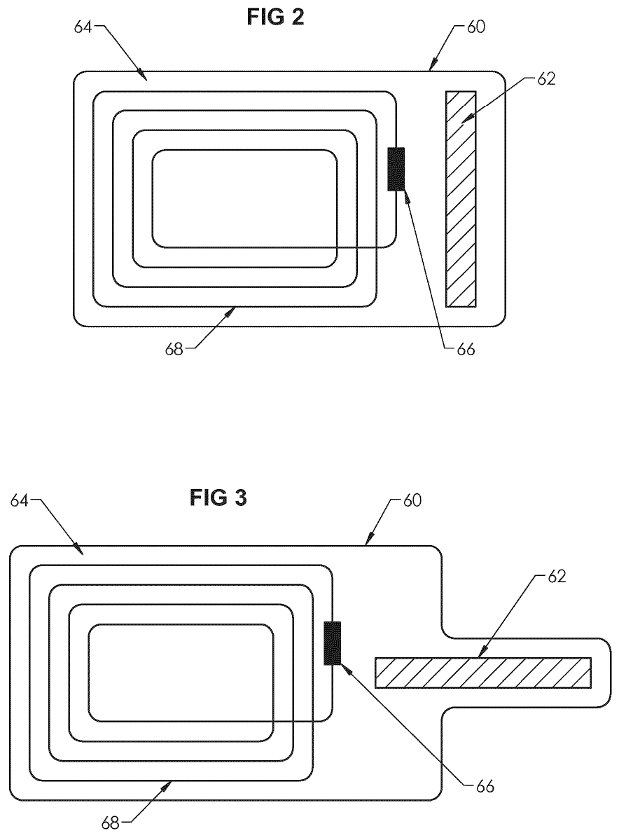

FIG. 1 shows a block diagram of an illustrative embodiment of elements of the surgical sponge counting and detection system 100 of the present invention. Sponges 2, 4, 6 and 8 may be received in the sponge entry detection zone 16 sequentially or simultaneously. One or more antennas 24, 26 and 28 may be operatively associated with (e.g. coupled to) the sponge entry detection zone 16 to detect and read data from the identifiers. The identifiers will be further discussed with reference to FIGS. 2-9. The antennas 24, 26, and 28 may establish a field to facilitate determining the identity of each sponge 2, 4, 6, 8 entering the sponge entry detection zone 16.

The sponge entry detection zone 16 is incorporated into a sponge entry recess 20. The sponge entry recess 20 may be formed as a recess for receiving sponges 2, 4, 6, 8. The data received by the antennas 24, 26, and 28 of the sponge entry detection zone 16 may be delivered to control circuitry 30 which may record data regarding each sponge 2, 4, 6, 8 entering the sponge entry detection zone 16. The control circuitry 30 may also function as a controller to provide energizing power to antennas 24, 26 and 28. The control circuitry 30 may control the operation of the counting and detection system 100 (“the system 100”) including various parameters and in the proper sequence to permit substantially simultaneous identification of a plurality of sponges 2, 4, 6, 8. A visual display 34 may be controlled by control circuitry 30 and may provide a visual display 34 with whatever information is desired regarding each sponge 2, 4, 6 and 8 being introduced into the sponge entry detection zone 16.

In various embodiments, the system 100 may employ a plurality of antennas at each of the sponge entry detection zone 16 and the sponge exit detection zone 40, such as the three antennas 24, 26 and 28 and the three antennas 42, 44, 46 shown in FIG. 1. However, in some embodiments the system 100 may alternatively be employed with a single antenna in each of the sponge entry detection zone 16 or exit detection zone 40. Identifier orientation with respect to the antenna may then become important. In this latter case, it may be beneficial to place the identifier of the sponge 2, 4, 6, 8 flat against the antenna. The manufacturer could create the identifiers with a consistent desired known flat configuration to facilitate uniform size, shape and positioning within a single or multiple packages of sponges intended for use in a single antenna system 100.

In some embodiments it may be advantageous to have the antenna(s) 24, 26, 28 or 42, 44, 46 move relative to the sponge entry recess 20 or sponge exit detection zone 40. In some embodiments the sponge entry recess 20 or the sponge exit detection zone 40 may move relative to the antenna(s) 24, 26, 28 or 42, 44, 46. In both of these embodiments, the movement increases the probability that the spatial orientation of the tag 60 relative to the antenna, will align properly to allow reading of the tag 60.

In operation, the sponges 2, 4, 6 and 8 may be delivered either manually or by another means, from the sponge entry detection zone 16 into the surgical field 36. At that point, the control circuitry 30 may have a record of each specific sponge 2 that has entered into the surgical field 36. The user may detect one sponge 2 at a time or a plurality of sponges simultaneously. If the user desires to detect only one sponge at a time, the system 100 may produce an error message if more than one sponge is detected simultaneously.

Sponges 2, 4, 6, 8 that are employed within the surgical field 36 may be introduced into the sponge exit detection zone 40 after use. The sponge exit detection zone 40 may be coupled with a plurality of antennas 42, 44 and 46. The antennas 42, 44, and 46 may generate a detection field with the sponge exit detection zone 40 such that specific identification of each identifier on each sponge 2 is provided. This may be accomplished in a manner to be described hereinafter for a plurality of sponges simultaneously. The data received may be delivered to control circuitry 30 for comparison and storage therein with the output being shown on the visual display 34. The control circuitry 30 may determine and account for the sponges 2, 4, 6, 8, and provide an indication as to whether the sponges 2 have been removed from the patient based on the data. The visual display 34 may be provided with controls which may permit simultaneous display of sponge entry data and sponge exit data as well as other desired information.

A single sponge or package of sponges may be introduced into the sponge entry detection zone 16, which may be provided with a single antenna or a plurality of antennas, such as antennas 24, 26 and 28. It may not be necessary to remove the individual sponges from the package in order to have the identifier of each sponge read. Two or more antennas 24, 26, 28 may be used so that the orientation of the sponges and, therefore, the orientation of the identifiers need not be controlled with respect to the antennas 24, 26, 28.

If desired, the sponge entry detection zone 16, rather than being a separate structure or area, may be provided at the discharge end of an object dispenser such as a sponge dispenser. In these embodiments, the system 100 may function as with the sponge entry detection zone 16 and may have withdrawal of the sponge from the storage container or dispenser serving as the act of triggering detection.

In some embodiments, the antennas 24, 26 and 28 of the sponge entry detection zone 16 may be the same antennas 42, 44 and 46 serving the sponge entry recess 20 and sponge exit detection zone 40 which may be the same zone. In this instance, the control circuitry 30 may need to be manually triggered to be informed that new sponges 2, 4, 6, 8 are entering the system 100. Alternately, new previously unidentified sponges 2, 4, 6, 8 may automatically be regarded by the control circuitry 30 as new sponges 2, 4, 6, 8 to the system 100.

In some embodiments, the function and data integrity of identifiers 62 or 64 may be verified at the sponge 2 entry and exit detection zones. For example, when a sponge 2, 4, 6, 8 enters the sponge entry detection zone 16 or the exit detection zone 40, the control circuitry 30 may, be used to compare the data read from the sponge's identifier 62 or 64 with data pre-programmed into control circuitry 30. If the data on the sponge's identifier 62 or 64 does not match the data pre-programmed into the control circuitry 30, the user may be alerted. This may ensure that all identifiers 62 or 64 are functional. In another embodiment, control circuitry 30 may be programmed to detect identifiers 62 or 64 that are malfunctioning, such as identifiers 62 or 64 that may have become damaged during shipping or a package identifier that may have been manufactured with an incorrect number of sponges 2, 4, 6, 8. The user may be alerted about the occurrence of such malfunctioning identifiers 62 or 64. This may for example prevent the incorrect counting of sponges 2, 4, 6, 8 or other errors.

In some embodiments of this invention, there is no need for a package identifier such as information on the IC chip, as part of the data transmitted from any given sponge 2, identifying information such as how many sponges 2, 4, 6, 8 are included in the given package because all of the sponges 2, 4, 6, 8 manufactured for use with this device will be supplied only in packages of a fixed number, such as non-consecutive numbers such as five and ten. Therefore, when a pack of sponges 2 is detected in the sponge entry detection zone 16, any sponge count other than the fixed number, such as five and ten, will be automatically rejected. Eliminating the package identifiers as information on the IC chip frees chip space for other information and results in faster interrogation because there is less data to interrogate. In the event that the operator is purposely entering sponges into the system 100 in counts other than five or ten, the counter may have a manual over-ride switch to allow the different number of sponges 2 to be entered.

In some embodiments, where multiple antennas such as antennas 24, 26 and 28 or 42, 44 and 46 cooperate with respect to a particular detection zone 16 or 40, it may be useful if the antenna's function as individuals, with the control circuitry 30 controlling the sequence and manner in which each antenna will function. For example, the control circuitry 30 may be structured to tune and detune the antennas within a group (24, 26 and 28) (42, 44 and 46) such that during a period of operation of one said antenna within a group, it is tuned to a specific resonant frequency, the remaining antennas of the group are detuned with respect to that frequency. Power may be supplied to only the antenna being tuned.

In other embodiments, one antenna (e.g., one of 24, 26, 28, 42, 44, 46) may be powered to a specific resonant frequency during a period when the others are un-powered. In still other embodiments, one antenna may be caused to emit a signal out-of-phase with respect to the others. Mechanical movement of the antennas 24, 26, 28, 42, 44, 46 relative to the sponges 2, 4, 6, 8 may be employed. Mechanical movement of the sponges 2, 4, 6, 8 relative to any of the antennas 24, 26, 28, 42, 44, 46 may also be employed.

In some embodiments, two RF detection technologies are advantageously used in parallel to detect retained sponges. For example, a single tag 60 on a sponge 2, 4, 6, 8 may include a sophisticated and relatively expensive UHF-RFID identifier 64 for detection and counting outside the body and a very inexpensive EAS identifier 62 for detection within the body. Alternately, the UHF-RFID identifier 64 and the EAS identifier 62 may be in two separate tags 60, both attached to the same sponge 2. While the two tags 60 increase the manufacturing cost of the sponge 2 slightly, they also increase the geographical distance between the two identifiers 62, 64, which reduces the probability of the two identifiers 62, 64 interfering with each other or “coupling.” In some embodiments, an HF-RFID identifier 64 could be combined with an EAS identifier 62.

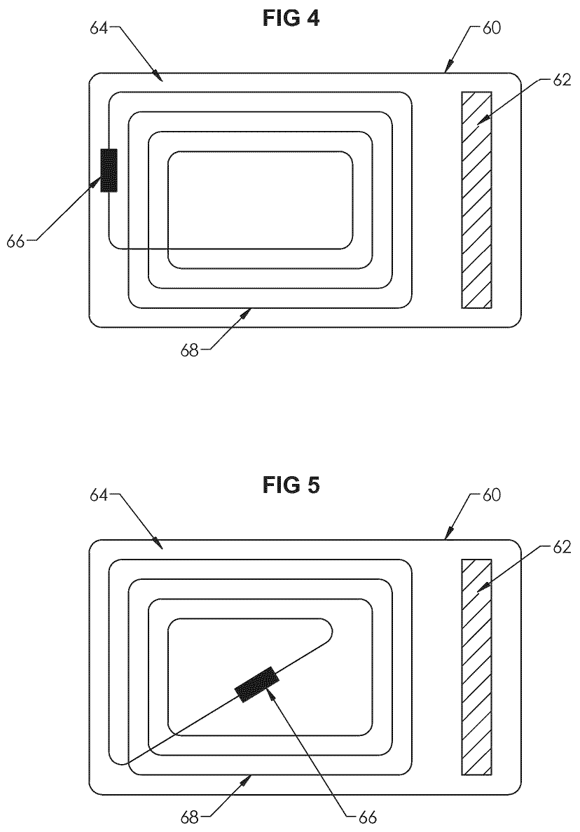

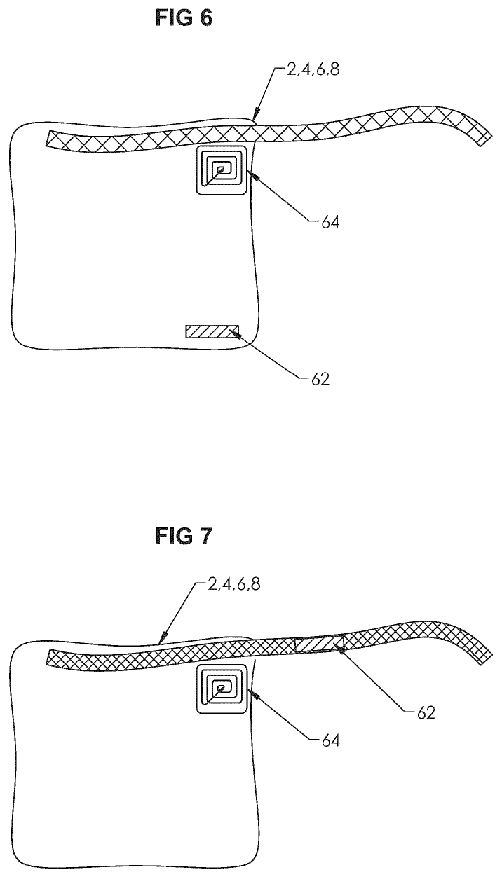

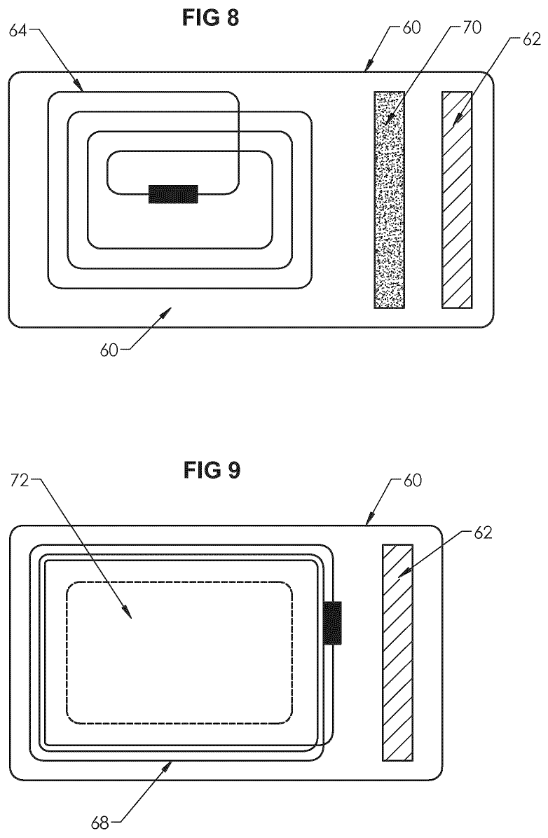

In some embodiments, the design of the tags 60 and sponges 2, 4, 6, 8 that simultaneously incorporate a uhf-rfid identifier 64 or HF-RFID identifier 64 and EAS identifier 62, must minimize the chances of the adjacent identifiers from “coupling” and canceling each other out, thus becoming undetectable. The two RF identifiers may be maximally separated geographically in some embodiments, either on the same tag 60 or on the same sponge 2. In some embodiments, the EAS identifier 62 may be maximally separated from the antenna portion 68 of the UHF-RFID identifier 64 as shown in FIGS. 2 and 3. Alternately, the EAS identifier 62 may be maximally separated from the IC portion 66 of the UHF-RFID identifier 64 as shown in FIGS. 4 and 5. In some embodiments, the EAS identifier 62 may be maximally separated from the UHF-RFID identifier 64 by attaching each identifier tag 60 to a separate location on the sponge 2, 4, 6, 8 as shown in FIGS. 6 and 7. In some embodiments, the two adjacent antennas may be oriented so that they emit their signals in parallel, perpendicularly away from the tag 60 and not toward each other. In some embodiments, a ferrite or metal foil strip 70 or other appropriate material may be interposed between the UHF-RFID identifier 64 and the EAS identifier 62, in order to dampen stray RF signals that may pass between the two identifiers, as shown in FIG. 8.

In some embodiments, the two adjacent RF identifiers are also maximally separated in their respective operating frequencies. For example, the frequency difference between EAS and UHF is greater than the frequency difference between EAS and HF. Therefore the combination of EAS and UHF may provide benefits over the combination of EAS and HF, in preventing interference between the adjacent identifiers.

In some embodiments, the operation of the two adjacent RF identifiers is separated temporally. For example, the control circuitry 30 may prevent simultaneous interrogation of the EAS and UHF-RFID identifiers 62, 64. Therefore, they are less likely to cause interference with each other.

In some embodiments, the UHF-RFID identifier antenna 68 may surround a hole 72 cut into the tag 60 material. The hole 72 may include an opening 72 or aperture extending from one surface of the tag 60 material through to an opposite surface of the tag 60 material. The hole 72 makes the film tag 60 more flexible and less noticeable when it is embedded into a surgical sponge 2. The material may include a plastic film encapsulating the antenna as in FIG. 9. The plastic film may be absent except in the areas where the film is encapsulating the antenna traces. The hole 72 in the plastic film may be any shape but generally may be substantially round, oval, square or rectangular. The hole 72 in the tag 60 allows the tag 60 size and the antenna size to be much larger and yet the increased flexibility of the tag 60 is not annoying to the surgeon compared to an equal sized stiff tag 60. A large tag 60 of this construction may be sandwiched between two or more layers of gauze or cotton cloth in the construction of the surgical sponge 2, 4, 6, 8. A flexible tag 60 of this construction can also be rolled up so that it can be passed through a tube such as a laparoscope.

In some embodiments, the two RF detection technologies are essentially operating in parallel. Each of the RF technologies have different failure modes or fail for different reasons. For example, UHF and HF-RFID identifiers 64 are much more sensitive to mechanical damage compared to EAS identifiers 62 which are relatively robust and durable. Another example is that UHF and HF-RFID identifiers 64 can experience programming glitches, whereas the non-programmable EAS identifiers 62 cannot. Another example is that inadvertent contact with a magnetic field can disable an EAS identifier 62 but will have little or no effect on a UHF and HR-RFID identifier 64.

Since each RF technology has different failure modes or fails for different reasons, the failure rate of the combined system 100 is approximately the failure rate of each technology multiplied together. For example, if each failed 1% of the time, the combined failure rate would be 1%×1%=0.01%. This uniquely leads to failure rates that are orders of magnitude less than can be achieved with any single sensing technology.

These are not “backup systems” where the secondary system 100 is activated only when the primary system 100 fails. These two sensing technologies operate substantially simultaneously in parallel, resulting in a geometric 100× or more improvement in the reliability of surgical sponge 2, 4, 6, 8 detection.

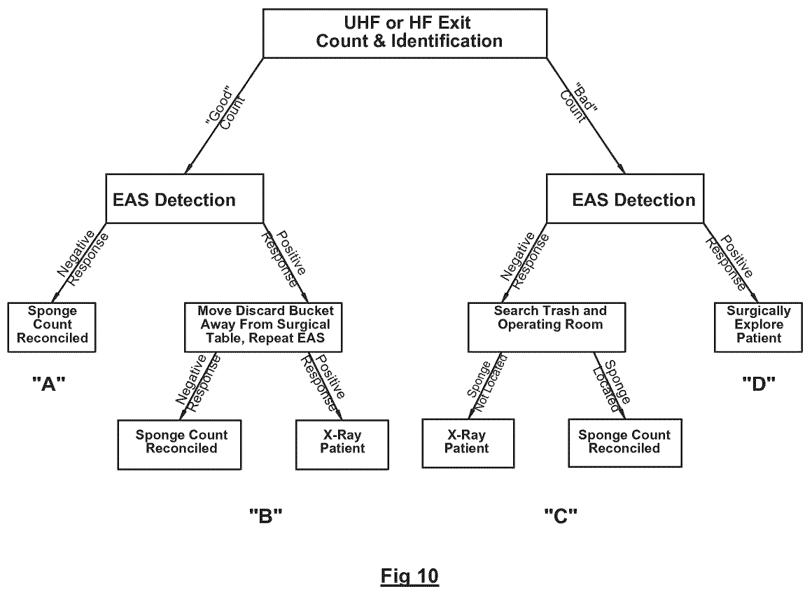

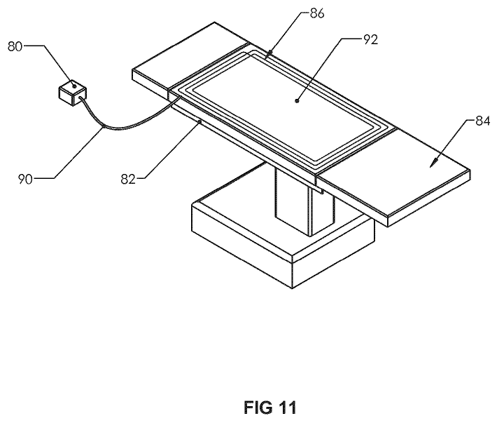

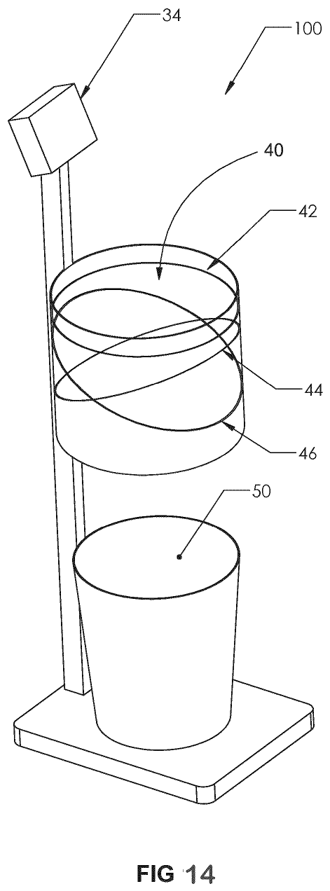

In some embodiments the two RFID technologies in the system 100 are a UHF-RFID system (FIGS. 1, 14) for counting and identification outside the body and an EAS system (FIGS. 11-13) for detection of sponges 2, 4, 6, 8 retained within the body (according to the decision tree of FIG. 10. However, in some embodiments a single HF-RFID identifier 64 could be interrogated by two separate readers creating a substantially parallel detection system 100. For example, a sophisticated HF-RFID reader/counter (FIG. 1, 14) may be used to detect and count sponges 2, 4, 6, 8 outside the patient's body and a much less sophisticated detector (FIGS. 11-13) without counting and identification capabilities may be embedded in the underbody support 84. Both of these detectors would be interrogating the same HF-RFID identifier 64 but operating substantially in parallel.

The reliability rate can be even further improved by adding an algorithm to analyze the input data from both RFID sources. For example, one common failure mode for EAS detection technology is that two identifiers located adjacent to each other can cause “coupling.” Coupling means that the signals from each EAS identifier 62 can cancel out the signal from the adjacent identifier (e.g., 64), resulting in no signal returning to the receiving antenna (e.g., 86, 88) despite two sponges 2, 4, 6, 8 being retained within the patient. In the instant invention with parallel RFID technologies, the UHF or HF counting technology would have to have miscounted two sponges to have two sponges unknowingly retained and coupled in the patient. If for example the failure rate for UHF-RFID counting is 1%, the chances of failing twice would be 0.01%. The additional 1% failure rate of the EAS detection technology would make the total failure probability of 1%×1%×1%=0.0001%. Alternately, if a single sponge 2 is missing according to the UHF counter, the failure rate of the EAS detector may be less than 1% because coupling cannot occur.

In normal operation, the new surgical sponges 2, 4, 6, 8 may be counted and individually identified while still in a pack of a predetermined, fixed number 5 or 10 pack. The pack of sponges which includes sponges 2, 4, 6, 8 for example is placed into the sponge entry detection zone 16 of the counting device of system 100. The data from the UHF identifiers 64 on each sponge 2 is stored in the control circuitry 30 and the new sponges 2 are placed into the sterile surgical field 36. As the used sponges are discarded from the sterile surgical field 36, they are placed in the sponge exit detection zone 40 where they are once again counted and individually identified. The sponge entry detection zone 16 and the sponge exit detection zone 40 of the counting device of system 100 may be the same location and utilize the same antenna(s) 24, 26, 28 or 42, 44, 46. As shown in FIGS. 1 and 14, after counting and identification in the sponge exit detection zone 40, the used sponges drop into the sponge disposal bucket 50 below to await disposal at the end of the procedure. At the end of the procedure, any unused sponges still in the sterile surgical field 36 may be placed into the sponge exit detection zone 40 for counting and identification and then dropped into the sponge disposal bucket 50. The input and output counts and the individual sponge 2 identities are then compared by the control circuitry 30. If the input and output data matches, the count matches or is a “good” count, which is indicated on the visual display unit. In some embodiments, at the end of the procedure, the EAS transponder 80 of the underbody support 84 (e.g., mattress 84) is energized even if the count is “good” and a detection sweep of the sterile surgical field 36 and the patient's body is accomplished. The EAS transponder 80 may also be energized at other times in the surgical procedure. The EAS transponder 80 may include cable 90.

The results of the counting and identification process and the EAS detection process are then introduced by the control circuitry 30 into an algorithm for analysis. One embodiment of a decision tree for rectifying a sponge 2, 4, 6, 8 miscount is shown in FIG. 10. In some embodiments, the control circuitry 30 in the counting and detection system 100 will automatically give visual or audible prompts to the surgical staff for the most logical sequence for reconciling a miscount condition. The algorithm uses the input data from both the UHF (or HF) counting and identification, and the EAS detection to quickly lead to the most efficient pathway to assure that no sponge 2 is inadvertently retained in the patient at the end of surgery. Additionally, the goals of some embodiments of this system 100 are:

1. Totally eliminate the need for hand counting of the sponges by the nursing staff, which eliminates a time consuming, potentially dangerous and distasteful job. It also eliminates the human error that is inevitable with hand counting.

2. Minimize the need for x-rays for documenting that a sponge is not left in the patient or documenting that the sponge is in the patient before surgical re-exploration is started. X-rays in the operating room are time consuming and expensive since the entire surgical staff may stand around for up to one hour while the x-ray is taken and processed and read.

3. Minimize the need for searching the trash and the operating room, either by hand or by a hand-held scanning antenna using either UHF (or HF), or EAS or both. UHF is an appropriate technology for scanning the room and trash because of its' relatively long read range.

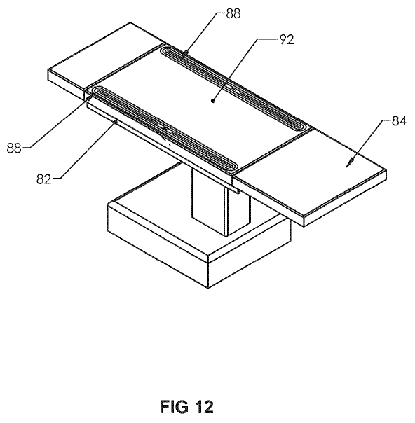

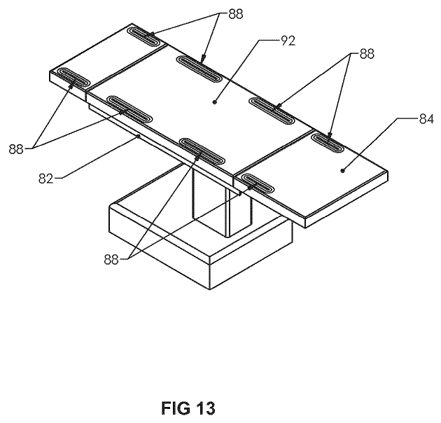

Referring to FIG. 10, if the UHF count is “good” meaning that the input and exit counts matched, indicating that no sponges are missing and the EAS detection has a “negative” response, meaning that no response signal was detected when the EAS antennas 86, 88 (FIG. 11 or 12) in the underbody support 84 were energized (Pathway “A”), the over-whelming odds are that no sponges are left in the patient, the operation is finished and no hand counting or x-rays are necessary.

Referring to FIG. 10, if the UHF count is “bad” meaning that the input and exit counts do not match indicating a missing sponge, and the EAS detection has a “positive” response, meaning that a response signal was detected when the EAS antennas 86, 88 (FIG. 11 or 12) in the underbody support 84 were energized (Pathway “D”), the over-whelming odds are that a sponge has been inadvertently left in the patient, and the patient should be immediately surgically explored. The probabilities of this being the situation are high enough that there is no need for time-consuming x-ray confirmation before surgically exploring the patient to retrieve the missing sponge(s).

Referring to FIG. 10, if the UHF count is “good” meaning that the input and exit counts matched indicating that no sponges 2 are missing, and the EAS detection has a “positive” response, meaning that a response signal was detected when the EAS antennas (e.g., 86, 88) in the underbody support 84 were energized (Pathway “B”), the input data from the two parallel systems are at odds with each other. This combination may indicate that the package of sponges must have had an extra sponge inserted during the manufacturing process, resulting in either 6 or 11 sponges in the pack instead of 5 or 10, for example. Additionally, one of the sponges 2, 4, 6, 8 may have a UHF (or HF) identifier 64 that has failed so that one sponge is silent and therefore the package was read during input to the system 100 as having the correct 5 or 10 count. Finally, the sponge retained in the patient may be the sponge with the UHF identifier 64 that has failed leaving the EAS identifier 62 still functional. Needless to say, the odds of this scenario are very low. The odds are much greater that the “positive” EAS response signal is due to a stray EAS signal, most likely from one of the sponges 2, 4, 6, 8 in the sponge disposal bucket 50. Therefore, the counting system 100 may give instructions, such as on the visual display 34, to move the sponge disposal bucket 50 further away from the surgical table and re-interrogate the EAS antennas 86 or 88 in the underbody support 84. If the “positive” EAS signal was a stray from the sponge disposal bucket 50, it should cease with moving the sponge disposal bucket 50 resulting in a “negative” EAS response and the sponge 2, 4, 6, 8 count is now reconciled. If the EAS signal is still present, move immediately to x-raying the patient to verify that the exceedingly low probability of a retained sponge in this scenario did not occur. If the x-ray is “negative,” ignore the “positive” EAS response.

Referring to FIG. 10, if the UHF count is “bad” meaning that the input and exit counts do not match indicating a missing sponge, and the EAS detection was a “negative” response, meaning that no response signal was detected when the EAS antennas 86, 88 in the underbody support 84 were energized (Pathway “C”), it is most likely that the missing sponge is not in the patient, however, the missing sponge must still be found and identified. Therefore, the next step is to move immediately to hand-held “wanding” or visual inspection of the trash and operating room as may be shown on the visual display 34. However, the low rate of EAS failure allows the surgeon to move forward with closing the incision while the missing sponge is being search for, saving both surgical and operating room time. If the missing sponge is found, the count is reconciled. If the missing sponge 2 is not found, move to x-raying the patient to confirm that the “negative” EAS response is accurate and that the missing sponge is not in the patient.

It is evident in FIG. 10 that there are no scenarios that lead to the need for the nurses to do hand counting of sponges with the parallel counting, identification and detection system 100 of this invention. The decision pathways leading to a reconciled, accurate count (A) or the need for surgical exploration (D) are quick and straight forward, saving time and money. Further, the decision tree minimizes the need for time-consuming and expensive x-rays in the operating room. Finally, only one relatively rare decision pathway (C) leads to the need to search the room and the trash, which is also inconvenient and time-consuming. However, this sponge counting and detection system 100 may include a handheld antenna to aid in the search of the room and trash. In addition, the users may be guided through the steps via the visual display 34.

In some embodiments as in FIG. 11, the antenna 86 for the EAS detector for detection within the body or the unsophisticated HF detector for detection within the body, is embedded in the underbody support 84, such as in mattress 84 of the surgical table 82. Embedding the antenna 86 in the underbody support 84 may be more convenient because it can eliminate the mattress 84 overlays that require extra cleaning. Embedding the antenna 86 in the underbody support 84 also can eliminate the user variability caused by hand-held antennas and the practice of “wanding”-waving a portable antenna over the patient in a generally uncontrolled, random fashion in order to detect a missing sponge. Using two or more antennas 88 (FIG. 12-13) that may be scanned using non-optical means may also ensure a reading regardless of the depths of sponges within the body of the patient. For example, each antenna 88 may be positioned at a different location in the underbody support 84, extending along the body of the patient, thereby ensuring that a wide reading range is covered.

Although the present embodiment incorporates embedding the antenna 86, 88 in the underbody support 84, in some embodiments, the features of the invention could alternatively incorporate the antenna 86, 88 into a mattress 84 overlay or other suitable component.

In some embodiments, the radiolucency of the central region of the underbody support 84 is maintained by either restricting the antennas 86, 88 to the periphery of the underbody support 84 or using radiolucent conductors. In some embodiments the one or more antennas 86, 88 are located near the edges of the mattress 84 in order to avoid disrupting x-ray pictures taken through the patient and mattress 84. The antenna may be a single large antenna 86 substantially surrounding a portion or all of the underbody support mattress 84 as in FIG. 11, or may be multiple smaller antennas 88 as in FIGS. 12 and 13. The advantage of multiple antennas 88 is the overlapping emission fields and emission angles that they create may be more likely to energize the antenna of the tag 60, which may be positioned at any random angle.

In some embodiments, the underbody support mattress 84 includes heater 92 material that is made of polypyrrole coated onto cloth. Polypyrrole is a material that is known for its ability to absorb RF signals such as in radar. Absorbing the stray RF signals that are known to be in the vicinity of the mattress 84 antennas 86, 88 advantageously improves the detection and clarity of the signal emitted from the tag 60.

Various embodiments may include one or more features described in U.S. patent application Ser. No. 13/422,279, Heated Under-body Warming Systems, filed Mar. 16, 2012, and U.S. Provisional Application No. 61/453,311, Heated mattress 84 and Heated mattress 84 Overlay for Therapeutic Under-body Warming, filed Mar. 16, 2011, both of which are incorporate by reference in the entirety. The embodiments may further include one or more features described in U.S. Provisional Patent Application 61/812,987, Flexible Electric Heaters, filed Apr. 17, 2013, which is also incorporated by reference in the entirety.

In some embodiments, the EAS reader communicates with the control circuitry 30 of the counting device of system 100 such as by a wired communication or by a wireless communication such as Bluetooth technology. The EAS reader and/or the counting device of system 100 may also communicate with other equipment in the vicinity of the surgical site (e.g., field) in order to briefly deactivate any equipment that may be emitting stray electrical and specifically RF signals. This may include equipment such as: the RF reader in the counting device of system 100, the power to the heater(s) 92 of the mattress 84, the RF electrosurgical device, and the pumps in an underbody support 84 that uses air, among other things. In some embodiments there is no physical connection between the EAS reader, which in some embodiments may be located near the surgical table, and the main counting device of system 100 which may be located away from the surgical table. There may be no cables that need to be plugged in which may risk tripping the surgical staff.

In some embodiments, data input to the counting device (e.g., apparatus,) of the system 100 may occur remotely or wirelessly from the rest of the counting system 100. Inputting data and controlling the counting device remotely separates the user and the controls from the bloody sponges 2. In a remote location, the user may not need to be wearing rubber gloves, which may interfere with touch screen inputs and contaminate the control surface with blood. A remote data input device such as a Bluetooth connected console or a “smart phone” or other device with a custom app, allows the controls on the counting device components to be limited to simple, easy to clean membrane switches that can be activated through a plastic protective drape, for example. Features of the counting device of the system 100 are further described with reference to FIG. 14.

In some embodiments, as in FIG. 14, the sponge exit detection zone 40 is separated from the sponge disposal bucket 50. In one embodiment, the sponge exit detection zone 40 is positioned above the sponge disposal bucket 50 with an open space of 6 inches-36 inches there between. When in use, the used sponges 48 are placed into the sponge exit detection zone 40 where the antennas 42, 44 and 46 interrogate the identifiers 62, 64 of the sponges 2, 4, 6, 8. When the identification is complete, the sponges 2, 4, 6, 8 may drop into the sponge disposal bucket 50 located below the sponge exit detection zone 40.

In some embodiments, the sponges 2 are mechanically retained briefly in the sponge exit detection zone 40 so that the reader has adequate time to interrogate multiple sponges 2, 4, 6, 8. Many types of movable mechanical obstructions are anticipated, that may be placed inside the sponge exit detection zone 40 to slow or stop the fall of the used sponges through the sponge exit detection zone 40. In some designs, the mechanical obstruction may not simply open and close but might also produce an up and down or circular movement in the pile of sponges 2, 4, 6, 8 which changes the orientation of the tag 60 of an individual sponge with respect to the antennas 42, 44, 46 of the sponge exit detection zone 40, for optimal reading.

In some embodiments as in FIG. 14, the used sponges 48 then fall from the sponge exit detection zone 40 into the sponge disposal bucket 50 below. The physical separation of the sponge exit detection zone 40 from the sponge disposal bucket 50 reduces the likelihood that the antennas 42, 44, 46 from the sponge exit detection zone 40 will continue to interrogate the sponges in the sponge disposal bucket 50 and if a sponge in the sponge disposal bucket 50 is interrogated, reduces the likelihood that the stray response signal will be detected by the antennas 42, 44, 46 in the sponge exit detection zone 40. In other words, when a sponge 2, 4, 6, 8 has been successfully interrogated and accounted for, it is placed in the isolated sponge disposal bucket 50 where it no longer interferes with the ongoing interrogation of subsequent sponges 2. In some embodiments, a disposable sponge disposal bag such as a plastic film bag extends from the base of the sponge disposal bucket 50, up and through the top of the sponge exit detection zone 40, bridging the open space between the sponge exit detection zone 40 and the sponge disposal bucket 50.

In some embodiments, the antennas 42, 44, 46 of the sponge exit detection zone 40, and/or the sponge disposal bucket 50 are physically surrounded by one or more layers of RF absorbing material such as polypyrrole coated cloth. In some embodiments, the antennas of the sponge exit detection zone 40 and/or the sponge disposal bucket 50 are physically surrounded by one or more layers of RF reflecting material such as metal foil. In some embodiments, the sponge disposal bucket 50 is made of, or physically surrounded by or coated with, one or more layers of ferrous metal or magnetic material to inactivate the EAS tags in the bucket. The polypyrrole cloth and/or metal foils or other material surrounding the sponge exit detection zone 40 may reduce the interference from other RF producing equipment such as the electro-surgical unit. The polypyrrole cloth and/or metal foils or other material surrounding the sponge exit detection zone 40 may also reduce the broadcast of stray RF signals throughout the room and the detection of sponge identifiers 62, 64 still located in the sterile surgical field 36. This is especially important with UHF because it has a significantly longer read-range than HF. Inadvertent detection of identifiers located outside the sponge exit detection zone 40 can occur with UHF and EAS.

In some embodiments, the optimum reading location of the antennas 24, 26, 28 in the sponge entry detection zone 16 may be visually indicated such as by intersecting beams of laser light. Therefore, the operator can place a package of sponges into the open space within the sponge entry detection zone 16 until the intersecting beams of light hit the package. At that point, the sponges are optimally located within the sponge entry detection zone 16 for optimal reading.

The systems provided by embodiments of the present invention may be used alone or in conjunction with other systems, methods, and apparatus. If the system 100 is used alone as a stand-alone device in a surgical field 36, it may be set up to communicate with other stand-alone devices. This may facilitate the compilation of information. Alternatively, a single stand-alone device may be adapted to store information across multiple surgical fields and to combine that information in a way the helps the user track the total number of sponges 2 in multiple surgical fields.

It will be appreciated that the present invention provides a number of efficient methods for effectively and accurately controlling the monitoring, detection, counting, identification and in some cases, further characterization of sponges 2, 4, 6, 8 entering and exiting a surgical field 36 or surgical site, so as to avoid inadvertent retention of a sponge within the a patient. In one embodiment, a patient is scanned with a lower array of antennas 88 underlying the patient to determine if any sponges 2, 4, 6, 8 are in the patient, particularly immediately prior to closing of the surgical incision at the end of the surgical procedure. In one embodiment, a sponge entry detection zone 16 and a sponge exit detection zone 40 cooperate to monitor the use of surgical sponges. In one embodiment, these two counting and detecting systems operate substantially simultaneously in parallel.

In the foregoing detailed description, the embodiments of the invention have been described with reference to specific embodiments. However, it may be appreciated that various modifications and changes can be made without departing from the scope of the embodiments of the invention as set forth in the numbered embodiments provided below.

Certain embodiments of the present invention are described in the following numbered illustrative embodiments:

1. A surgical sponge comprising:
an absorbent body;
a Radio Frequency Identification (RFID) identifier attached to the absorbent body;
an Electronic Article Surveillance (EAS) identifier attached to the absorbent body;
wherein the RFID and EAS identifiers are separate from each other, and the RFID and EAS identifiers have substantially different modes of failure causation.

2. A surgical sponge counting and detection system for counting and detecting surgical sponges for use in the treatment of a patient, the system comprising:
a Radio Frequency Identification (RFID) scanner adapted to detect entry and exit of a surgical sponge RFID identifier associated with each of a plurality of surgical sponges;
an Electronic Article Surveillance (EAS) scanner adapted to detect the presence of a surgical sponge EAS identifier associated with each of a plurality of surgical sponges;
a display unit for indicating information regarding the surgical sponges; and
a control circuit operably coupled to the RFID scanner, the EAS scanner, and the display unit, wherein the control circuit is configured to determine, based on the scan data from the RFID scanner, if the number of sponges entry scanned is equal to the number of sponges exit scanned, and to further determine, based on the scan data from the EAS scanner, if any sponges remain in the patient, further wherein the control circuit is configured to provide an indication, via the display unit, that all the sponges have been removed from the patient, or that one or more sponges still remain in the patient.

3. A method of surgical sponge counting and detection for counting and
detecting surgical sponges during surgery on a patient, the system comprising:
providing surgical sponges, each sponge comprising:
   an absorbent body;
   a Radio Frequency Identification (RFID) identifier attached to the absorbent body;
   an Electronic Article Surveillance (EAS) identifier attached to the absorbent body;
   wherein the RFID and EAS identifiers are separate from each other, and
   the RFID and EAS identifiers have substantially different modes of failure causation;
providing a sponge counting and detection system comprising
   an RFID scanner adapted to detect a surgical sponge RFID identifier associated with each of a plurality of surgical sponges;
   an EAS scanner adapted to detect a surgical sponge EAS identifier associated with each of a plurality of surgical sponges;
   a display unit;
   a control circuit operably coupled to the RFID scanner, the EAS scanner, and the display unit;
entry scanning the sponges entering the surgical field via the RFID scanner and sending the RFID entry scan data to the control circuit;
exit scanning the sponges exiting the surgical field via the RFID scanner and sending the RFID exit scan data to the control circuit;
scanning the space occupied by the patient via the EAS scanner and sending the EAS scan information to the control circuit;
determining, based on the entry and exit scan data, if the number of sponges that have entry scanned and exit scanned are equal,
determining based on the scan data from the EAS scanner if any sponges remain in the patient; and
providing an indication, via the display unit, that all the sponges have been removed from the patient, or providing an indication that one or more sponges still remain in the patient.

4. The surgical sponge of embodiments 1, 2 or 3, wherein the RFID identifier is UHF.

5. The surgical sponge of embodiments 1, 2 or 3 or any of the preceding embodiments, wherein the RFID identifier is HF.

6. The surgical sponge of embodiments 1, 2 or 3 or any of the preceding embodiments, wherein the RFID identifier is operable to communicate a unique alpha-numeric data string corresponding to the specific tagged sponge.

7. The surgical sponge of embodiments 1, 2 or 3 or any of the preceding embodiments, wherein the EAS identifier is magnetic or magneto-harmonic.

8. The surgical sponge of embodiments 1, 2 or 3 or any of the preceding embodiments, wherein the EAS identifier is acousto-magnetic or magnetostrictive.

9. The surgical sponge of embodiments 1, 2 or 3 or any of the preceding embodiments, wherein the EAS identifier is radio frequency or low frequency.

10. The surgical sponge of embodiments 1, 2 or 3 or any of the preceding embodiments, wherein the EAS identifier is microwave.

11. The surgical sponge of embodiments 1, 2 or 3 or any of the preceding embodiments, wherein the RFID identifier and the EAS identifier are operably separated from each other.

12. The surgical sponge of embodiments 1, 2 or 3 or any of the preceding embodiments, further comprising a tag, wherein the RFID identifier and the EAS identifier are geographically spaced apart from one another on the tag or the sponge.

13. The surgical sponge of embodiments 1, 2 or 3 or any of the preceding embodiments, wherein the RFID identifier and the EAS identifier are operating at frequencies that are separated from each other.

14. The surgical sponge of embodiments 1, 2 or 3 or any of the preceding embodiments, wherein the RFID identifier and the EAS identifier are temporally interrogated at times that are separated from each other.

15. The surgical sponge of embodiments 1, 2 or 3 or any of the preceding embodiments, wherein the RFID identifier and the EAS identifiers have substantially different modes of failure causation which leads to a combined failure rate that approximates the failure rate of the RFID identifier multiplied by the failure rate of the EAS identifier.

16. The surgical sponge of embodiments 1, 2 or 3 or any of the preceding embodiments, wherein the combined failure rate of the RFID and the EAS identifiers is approximately two orders of magnitude more reliable than the failure rate of the RFID identifier or the EAS identifier individually.

17. The surgical sponge counting and detection system of any of embodiments 2-16, wherein the RFID scanner is configured to interrogate a plurality of identification tagged sponges having an RFID identifier thereon, said scanner detecting each of the plurality of tagged sponges electromagnetically, said scanner detecting unique data strings corresponding to each of the plurality of tagged sponges.

18. The surgical sponge counting and detection system of any of embodiments 2-17, wherein the RFID scanner is configured to interrogate the plurality of RFID identification tagged sponges that are simultaneously present within a volume, the scanner simultaneously reading the plurality of tagged sponges present in the volume.

19. The surgical sponge counting and detection system of any of embodiments 2-18, wherein the RFID scanner is configured to interrogate the plurality of RFID identification tagged sponges as they enter the surgical field to establish an accurate count and specific sponge identification and to re-interrogate the plurality of RFID identification tagged sponges later as the same sponges exit the surgical field, to verify that all sponges entering the surgical field are documented as having exited the surgical field and are placed in the sponge disposal bucket.

20. The surgical sponge counting and detection system of any of embodiments 2-19, wherein the EAS scanner is configured to interrogate the identification tagged surgical sponges having an EAS identifier thereon.

21. The surgical sponge counting and detection system of any of embodiments 2-20, wherein the EAS scanner is configured to interrogate a plurality of identification tagged sponges having an EAS identifier thereon while they are within the human body.

22. The surgical sponge counting and detection system of any of embodiments 2-21, wherein the EAS scanner includes one or more antennas that are embedded in the underbody support to interrogate a plurality of identification tagged sponges having an EAS identifier thereon.

23. The method of surgical sponge counting and detection of embodiment 3 or any of the preceding embodiments, wherein if the RFID count is “good” meaning that the input and exit counts matched, indicating that no sponges are missing and the EAS detection has a “negative” response, meaning that no response signal was detected when the EAS antennas in the underbody support were energized, the over-whelming odds are that no sponges are left in the patient, the operation is finished and no hand counting or x-rays are necessary.

24. The method of surgical sponge counting and detection of embodiment 3 or any of the preceding embodiments, wherein if the RFID count is “bad” meaning that the input and exit counts do not match indicating a missing sponge, and the EAS detection has a “positive” response, meaning that a response signal was detected when the EAS antennas in the underbody support were energized, the over-whelming odds are that a sponge has been inadvertently left in the patient, and the patient should be surgically explored.

25. The method of surgical sponge counting and detection of embodiment 3 or any of the preceding embodiments, wherein if the RFID count is “good” meaning that the input and exit counts matched indicating that no sponges are missing, and the EAS detection has a “positive” response, meaning that a response signal was detected when the EAS antennas in the underbody support were energized, the odds are that the “positive” EAS response signal is due to a stray EAS signal, most likely from one of the sponges in the sponge discard bucket which should be moved further away from the surgical table and the EAS antennas in the underbody support re-interrogated and if the EAS signal is still present, move immediately to x-raying the patient to verify that the exceedingly low probability of a retained sponge in this scenario did not occur.

26. The method of surgical sponge counting and detection of embodiment 3 or any of the preceding embodiments, wherein if the RFID count is “bad” meaning that the input and exit counts do not match indicating a missing sponge, and the EAS detection was a “negative” response, meaning that no response signal was detected when the EAS antennas in the underbody support were energized, the odds are that the missing sponge is not in the patient, however, the missing sponge must still be found and identified by moving immediately to hand-held “wanding” or visual inspection of the trash and operating room.

27. The method of surgical sponge counting and detection of embodiment 3 or any of the preceding embodiments, wherein the low rate of EAS failure allows the surgeon to move forward with closing the incision while the missing sponge is being search for, saving both surgical and operating room time.

Whereas particular embodiments of the invention have been described herein for the purposes of illustration, it will be evident to those skilled in the art that numerous variations and/or combinations of the details may be made without departing from the invention.

1. A surgical sponge comprising:
an absorbent body;
a Radio Frequency Identification (RFID) identifier attached to the absorbent body;
an Electronic Article Surveillance (EAS) identifier attached to the absorbent body;
wherein the RFID and EAS identifiers are separate from each other, and
the RFID and EAS identifiers have substantially different modes of failure causation.

2. The surgical sponge of claim 1, wherein the RFID identifier is UHF.

3. The surgical sponge of claim 1, wherein the RFID identifier is HF.

4. The surgical sponge of claim 1, wherein the RFID identifier is operable to communicate a unique alpha-numeric data string corresponding to the respective sponge.

5. The surgical sponge of claim 1, wherein the EAS identifier is magnetic or magneto-harmonic.

6. The surgical sponge of claim 1, wherein the EAS identifier is acousto-magnetic or magnetostrictive.

7. The surgical sponge of claim 1, wherein the EAS identifier is radio frequency or low frequency.

8. The surgical sponge of claim 1, wherein the EAS identifier is microwave.

9. The surgical sponge of claim 1, wherein the RFID identifier and the EAS identifier are operably separated from each other.

10. The surgical sponge of claim 1, further comprising a tag, wherein the RFID identifier and the EAS identifier are geographically spaced apart from one another on the tag or the sponge.

11. The surgical sponge of claim 1, wherein the RFID identifier and the EAS identifier are operating at frequencies that are separated from each other.

12. The surgical sponge of claim 1, wherein the RFID identifier and the EAS identifier are temporally interrogated at times that are separated from each other.

13. The surgical sponge of claim 1, wherein the RFID identifier and the EAS identifiers have substantially different modes of failure causation which leads to a combined failure rate that approximates the failure rate of the RFID identifier multiplied by the failure rate of the EAS identifier.

14. The surgical sponge of claim 13, wherein the combined failure rate of the RFID and the EAS identifiers is approximately two orders of magnitude more reliable than the failure rate of the RFID identifier or the EAS identifier individually.

15. A surgical sponge counting and detection system for counting and detecting surgical sponges for use in the treatment of a patient, the system comprising:
a Radio Frequency Identification (RFID) scanner adapted to detect entry and exit of a surgical sponge RFID identifier associated with each of a plurality of surgical sponges;
an Electronic Article Surveillance (EAS) scanner adapted to detect the presence of a surgical sponge EAS identifier associated with each of a plurality of surgical sponges;
a display unit for indicating information regarding the surgical sponges; and
a control circuit operably coupled to the RFID scanner, the EAS scanner, and the display unit, wherein the control circuit is configured to determine, based on the scan data from the RFID scanner, if the number of sponges entry scanned is equal to the number of sponges exit scanned, and to further determine, based on the scan data from the EAS scanner, if any sponges remain in the patient, further wherein the control circuit is configured to provide an indication, via the display unit, that all the sponges have been removed from the patient, or that one or more sponges still remain in the patient.

16. The surgical sponge counting and detection system of claim 15, wherein the RFID scanner is configured to interrogate a plurality of identification tagged sponges having an RFID identifier thereon, said scanner detecting each of the plurality of tagged sponges electromagnetically, said scanner detecting unique data strings corresponding to each of the plurality of tagged sponges.

17. The surgical sponge counting and detection system of claim 15, wherein the EAS scanner is configured to interrogate the identification tagged surgical sponges having an EAS identifier thereon.

18. The surgical sponge counting and detection system of claim 16, wherein the RFID scanner is configured to interrogate the plurality of RFID identification tagged sponges that are simultaneously present within a volume, the scanner simultaneously reading the plurality of tagged sponges present in the volume.

19. The surgical sponge counting and detection system of claim 16, wherein the RFID scanner is configured to interrogate the plurality of RFID identification tagged sponges as they enter the surgical field to establish an accurate count and specific sponge identification and to re-interrogate the plurality of RFID identification tagged sponges later as the same sponges exit the surgical field, to verify that all sponges entering the surgical field are documented as having exited the surgical field and are placed in the sponge disposal bucket.

20. A method of surgical sponge counting and detection for counting and detecting surgical sponges during surgery on a patient, the system comprising:
providing surgical sponges, each sponge comprising:
an absorbent body;
a Radio Frequency Identification (RFID) identifier attached to the absorbent body;
an Electronic Article Surveillance (EAS) identifier attached to the absorbent body;
wherein the RFID and EAS identifiers are separate from each other, and
the RFID and EAS identifiers have substantially different modes of failure causation,
providing a sponge counting and detection system comprising
an RFID scanner adapted to detect a surgical sponge RFID identifier associated with each of a plurality of surgical sponges;
an EAS scanner adapted to detect a surgical sponge EAS identifier associated with each of a plurality of surgical sponges;
a display unit;
a control circuit operably coupled to the RFID scanner, the EAS scanner, and the display unit;
entry scanning the sponges entering the surgical field via the RFID scanner and sending the RFID entry scan data to the control circuit;
exit scanning the sponges exiting the surgical field via the RFID scanner and sending the RFID exit scan data to the control circuit;
scanning the space occupied by the patient via the EAS scanner and sending the EAS scan information to the control circuit;
determining, based on the entry and exit scan data, if the number of sponges that have entry scanned and exit scanned are equal,
determining based on the scan data from the EAS scanner if any sponges remain in the patient; and
providing an indication, via the display unit, that all the sponges have been removed from the patient, or providing an indication that one or more sponges still remain in the patient.
